(19) United States
(12) Patent Application Publication
(10) Pub. No.: US 2019/0062711 A1
(43) Pub. Date: Feb. 28, 2019

(54) Use Of Phosphorylated TBeta4 And Other Factors To Generate Human Induced Pluripotent Stem Cells

(71) Applicant: So Young Life Sciences Corporation, San Dimas, CA (US)

(72) Inventor: Avinash Seth, Pomona, CA (US)

(21) Appl. No.: 16/044,142

(22) Filed: Jul. 24, 2018

Related U.S. Application Data
(60) Provisional application No. 62/550,337, filed on Aug. 25, 2017.

(51) Int. Cl. C12N 5/074; C07K 5/00

(52) U.S. Cl. CPC C12N 5/0696; C07K 5/00; C12N 2501/72; C12N 2501/602; C12N 2501/603

(57) ABSTRACT

The present invention provides compositions and methods for inducing pluripotency in non-embryonic and/or somatic cells using exogenous Tb4, exogenous Sox2, and exogenous Oct4. Induced pluripotent stem cells can be can be simian or murine. The exogenous Tb4 can be phosphorylated. At least one of the exogenous Tb4, the exogenous Sox2, and the exogenous Oct4 can be coupled with a cell membrane penetrating moiety and/or a nuclear targeting moiety, allowing them to reach to the nucleus. In addition, the induced pluripotent stem cell maintains pluripotency over at least 100 passages.

The present application claims the benefit of and priority to a pending Provisional Patent Application Ser. No. 62/550,337 filed on Aug. 25, 2017, and titled “use of phosphorylated T134 other factors to generate human induced pluripotent stem cells”, which is hereby incorporated fully by reference into the present application.

These and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

### FIELD OF THE INVENTION

The field of the invention is compositions and methods of producing human induced pluripotent stem cells.

### BACKGROUND

The following background discussion includes information that may be useful in understanding the present invention. It is not an admission that any of the information provided herein is prior art or relevant to the presently claimed invention, or that any publication specifically or implicitly referenced is prior art.

There is a need for cures for degenerative diseases, and embryonic stem cell implants have shown some promise. (Olle Lindvall & Zaal Kokaia, Stem cells for the treatment of neurological disorders, 441 NATURE 1094-96, 2006). However, use of embryonic stem cells is controversial. In 2006, Yamanaka et al. reported the generation of induced pluripotent stem cells from mouse embryonic fibroblasts using four transcription factors, Oct3/4, Sox2, c-Myc, and Klf4. Kazutoshi Takahashi & Shinya Yamanaka (Induction of pluripotent stem cells from mouse embryonic and adult fibroblast cultures by defined factors, 126 CELL 663-76, 2006). In 2007, these transcription factors were employed to induce pluripotent stem cells from human fibroblasts. (Kazutoshi Takahashi et al., Induction of Pluripotent Stem Cells from Adult Human Fibroblasts by Defined Factors, 131 CELL 861-72, 2007).

Induced pluripotent stem cells have similar properties to embryonic stem cells, and may also be developed into degenerative disease therapies. Additionally, to study disease progression, iPSCs (induced Pluripotent Stem Cells) can be generated by reprogramming somatic cells of healthy and diseased individuals. The iPSCs may then be genetically modified by introducing or correcting mutations suspected to cause diseases. The iPSCs are then differentiated into the cell type of interest. (Yu Fen Samantha Seah et al., Induced Pluripotency and Gene Editing in Disease Modelling: Perspectives and Challenges, 16 INT. J. MOL. SCI. 28614-34, 2015).

However, several problems have prevented iPSCs from being widely used in therapies. One problem is the formation of tumors, because the Yamanaka transcription factors promote tumorigenesis, such that implanted cells do not behave normally. Additionally, we cannot predict consequence due to permanent genome modification by use of viral vectors.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Thus, there is still a need for induced pluripotent stem cells that lack permanent genome modifications, and do not form tumors when implanted in patients.

### SUMMARY OF THE INVENTION

The inventive subject matter provides compositions and methods for inducing pluripotency in non-embryonic and/or somatic cells using exogenous Tβ4, exogenous Sox2, and exogenous Oct4. Induced pluripotent stem cells can be derived from non-embryonic or somatic cells. Contemplated induced pluripotent stem cells can be simian (e.g., human induced pluripotent stem cells) or murine (e.g., mouse induced pluripotent stem cells). The inventors further contemplate that pluripotentcy can be induced in cells derived from livestock. The use of any and all examples, or exemplary language (e.g., “such as”) provided with respect to certain embodiments herein is intended merely to better illuminate the invention and does not pose a limitation on the scope of the invention otherwise claimed.

Preferably, the exogenous Tβ4 is phosphorylated. Phosphorylated Tβ4 promotes pluripotency by activating the p53 pathway and suppressing Ras oncogenes and the JAK-STAT pathway.

To increase the transfection efficiency, a cell membrane penetrating moiety can be coupled with the exogenous Tβ4, the exogenous Sox2, and/or the exogenous Oct4. As used herein, the term “coupled with” means covalently bonded, electrostatic, aviden/streptavidin, etc. The coupling includes linkers and/or spacers. As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

Preferably, the cell membrane penetrating moiety comprises a cationic peptide. Cationic peptides include arginine and lysine, preferably a sequence consisting essentially of R9-K9 (RRRRRRRRRKKKKKKKKK). The cationic peptide may comprise one or more glycine spacers. Optionally, a flexible linker may link the cationic peptide to the Tβ4, Sox2, and/or Oc4. This discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

Coupling the exogenous Tβ4, the exogenous Sox2, and/or the exogenous Oct4 to a nuclear targeting moiety is likely to increase expression of the target genes. A preferred nuclear targeting moiety is glutathione-S-transferase.

The inventors contemplate that induced pluripotent stem cells according to the inventive subject matter will maintain pluripotency over at least 100 passages.

The inventive subject matter also includes a composition that comprises exogenous Tβ4, exogenous Sox2, and exogenous Oct4 that can induce pluripotency in non-embryonic/somatic cells. In contemplated compositions, at least two of the exogenous Tβ4, the exogenous Sox2, and the exogenous Oct4 are coupled by a linker. In other words, a linker couples Tβ4 and Sox2, Tβ4 and Oct4, or Sox2 and Oct4. Linkers could also be used to couple Tβ4, Sox2, and Oct4.

A cell membrane penetrating moiety (e.g., a cationic peptide) and/or a nuclear targeting moiety (e.g., glutathione-S-transferase) can be coupled to the exogenous Tβ4, the exogenous Sox2, and/or the exogenous Oct4. A nuclear targeting moiety can optionally is coupled to at least one of the exogenous Tβ4; the exogenous Sox2; and the exogenous Oct4.

The inventive subject matter further includes a method of inducing pluripotency in a somatic cell comprising steps of: (1) introducing into the somatic cell Tβ4, Sox2, and Oct 4 proteins; and (2) culturing the somatic cell from step (1) under conditions that induce de-differentiation in the somatic cell, wherein an induced pluripotent stem cell is obtained. The inventive method can further include a step of culturing the induced pluripotent stem cell under conditions such that the induced pluripotent stem cell maintains pluripotency after 100 passages.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a schematic of a method according to the inventive subject matter.

### DETAILED DESCRIPTION

Induced pluripotent stem cells (iPSCs) have the potential to treat diseases such as, diabetes, Alzheimer's disease, Parkinson's disease, cardiovascular disease, and amyotrophic lateral sclerosis. Because iPSCs are generated from adult somatic cells, iPSCs could also be used for autologous cell replacement therapy and organ transplants. The inventors hypothesize that the inventive iPSCs will express cell surface markers including SSEA1, SSEA3, Sox2, Oct3/Oct4, Nanog, Klf4, c-Myc, and Lin28. The inventors further expect iPSCs according to the inventive subject matter to exhibit DNA methylation patterns and other epigenetic characteristics of the source cell.

The inventive subject matter provides compositions and methods for inducing pluripotency in non-embryonic and/or somatic cells using exogenous Tβ4, exogenous Sox2, and exogenous Oct4. The exogenous Tβ4, exogenous Sox2, and exogenous Oct4 are recombinant proteins used to reprogram non-embryonic stem cells including adult somatic cells. After induced pluripotent stem cells are obtained they can be differentiated into, for example, cardiomyocytes, adipocytes, dopaminergic neurons, neural cells, motoneurons, pancreatic β-cells, and hematopoietic progenitor cells. Such induced pluripotent stem cells have potential applications in disease modeling, drug screening, regenerative medicine, and cell therapy.

Induced pluripotent stem cells can be derived from non-embryonic or somatic cells from mammals from any order: Artiodactyla, Carnivora, Cetacea, Chiroptera, Dermoptera, Edentata, Hyracoidae, Insectivora, Lagomorpha, Marsupialia, Monotremata, Perissodactyla, Pholidata, Pinnipedia, Primates, Proboscidea, Rodentia, Sirenia, and Tubulidentata. Contemplated induced pluripotent stem cells can be simian (e.g., human induced pluripotent stem cells) or murine (e.g., mouse induced pluripotent stem cells). The inventors further contemplate that pluripotentcy can be induced in cells derived from livestock, such as, goats, cows, horses, and sheep.

In a preferred embodiment of the inventive subject matter, the somatic/non-embryonic cells comprise fibroblasts, cord blood cells (preferably CD34-positive), fibroblast-like synoviocytes, hepatocytes, gastric epithelial cells, B lymphocytes, pancreatic beta cells, keratinocytes, dental stem cells, mesenchymal stromal cells, peripheral mononuclear blood cells(preferably CD34-positive), and/or any other suitable cell. See e.g., Jun Li, Wei Song & Jun Zhou, Advances in understanding the cell types and approaches used for generating induced pluripotent stem cells, J. HEMATOLOGY & ONCOLOGY (2014), at 7:50 (https://doi.org/10.1186/s13045-014-0050-z).

Without wishing to be bound by a particular hypothesis, the inventor expects exogenous thymosin β4 (“Tβ4”) to induce pluripotency in non-embryonic and/or somatic cells, especially in combination with exogenous Sox2 and Oct4, while also suppressing tumorigenesis. Tβ4 is a 43 amino acid, 4.9 kDa protein present in all cell types (except erythrocytes). The many biological roles played by Tβ4 include binding to G-actin and depolymerization of F-actin into G-actin, which has been linked to cellular proliferation, differentiation, and migration. In cornea, Tβ4 has anti-inflammatory properties, suppresses apoptosis, and promotes cell migration and wound healing. Gabriel Sonse, Ping Qiu, Michelle Kurpakus-Wheater, Thymosin beta 4: A novel corneal wound healing and anti-inflammatory agent, 1(3) CLINICAL OPHTHALMOLOGY 201-07 (2007). Tβ4 has also been identified as a potential treatment for neurological injury, because Tβ4 mediates oligodendrogenesis and treats demyelination. Manoranjan Santra et al., Thymosin beta 4 mediates oligodendrocyte differentiation by upregulating p38 MAPK, 60(12) GLIA 1826-38 (2012).

There is precedent for Tβ4 to play a role in inducing pluripotency. Tβ4 has been shown to induce reversion of adult epicardium-derived progenitor cells to their embryonic phenotype. Paul R. Riley & Nicola Smart, Thymosin β4 induces epicardium-derived neovascularization in the adult heart. 37(6) BIOCHEM. SOC. TRANS. 1218-20 (2009). Riley and Smart suggested that the ability of synthetic Tβ4 to restore pluripotency may aid myocardial regeneration and neovascularization after acute ischemic injury. After Tβ4 induced pluripotency in epicardium-derived cells, the resulting induced pluripotent epicardium-derived cells underwent epithelial-mesenchyme transition, migrated away from the epicardium, and differentiated into vascular precursors that may contribute to neovascularization of the adult heart. The ability of Tβ4 to induce pluripotency is unpredictable in view of reports that Tβ4 plays a role in T-cell and endothelial cell differentiation.

Tβ4 has also been shown to have both tumor suppressive and tumorigenic properties. Jo Caers et al. reported that Tβ4 has tumor suppressive effects in multiple myeloma. Jo Caers et al., Thymosin β4 has tumor suppressive effects and its decreased expression results in poor prognosis and decreased survival in multiple myeloma. 95(1) HAEMATOLOGICA 163-67 (2010). Specifically, Tβ4 expression was down regulated in multiple myeloma cells, which was correlated with shorter survival times in mice. Boosting Tβ4 expression increased survival times. In contrast, overexpression of Tβ4 has been linked to increased growth, motility, and invasion in vitro and increased tumor load in vivo of solid tumors (e.g., colon cancer).

Preferably, the exogenous Tβ4 is phosphorylated to suppress oncogenic pathways. Exemplary Tβ4 amino acid sequences include UniProt database accession number P62328 (primary sequence) and conservatively modified variants thereof.

Meng et al. previously demonstrated that cord blood-derived CD34⁺ cells could be reprogrammed using a lentiviral vector that expresses Oct4 and Sox2 alone when the woodchuck post-transcriptional regulatory element and strong spleen focus-forming virus are included in the vector to increase expression. Xianmei Meng et al., Efficient Reprogramming of Human Cord Blood CD34⁺ Cells Into Induced Pluripotent Stem Cells With OCT4 and SOX2 Alone, 20(2) MOLECULAR THERAPY 408-16 (2012). The authors suggest that fibroblasts could be reprogrammed using a promoter that increases transgene expression in fibroblasts, such as EF1. Reprogramming could also be accomplished using weaker promoters of Oct4 and Sox2 expression in combination with a vector encoding KLF4 and Myc. However, as previously noted Myc is oncogenic.

The problem of reprogramming efficiency being limited by expression levels may be obviated by reprogramming cells directly with the proteins Oct4 and Sox2. Reprogramming is expected to be further enhanced by Tβ4, and preferably phosphorylated Tβ4. Exemplary amino acid sequences for Sox2 includes UniProt database accession number P48431 (primary sequence) and conservatively modified variants thereof. Exemplary amino acid sequences for Oct4 include UniProt database accession number Q01860(primary sequence) and conservatively modified variants thereof.

Using compositions and methods according to the inventive subject matter, the transfection efficiency is projected to be greater than 1%, 2%, 5%, and 10%. To further increase the transfection efficiency, and thus the reprogramming efficiency, a cell membrane penetrating moiety can be coupled with the exogenous Tβ4, the exogenous Sox2, and/or the exogenous Oct4. As used herein, and unless the context dictates otherwise, the term “coupled” is intended to include both direct coupling (in which two elements that are coupled to each other contact each other) and indirect coupling (in which at least one additional element is located between the two elements). Therefore, the terms “coupled to” and “coupled with” are used synonymously. For example, the exogenous proteins and peptides can be covalently bonded via peptide bonds, coupled via electrostatic interactions, coupled via affinity (e.g., biotin with avidin or streptavidin), etc. The coupling includes linkers and/or spacers such as G4S, polyethylene glycol, or other suitable linkers. ProteoChem™ offers several suitable heterobifuntional crosslinkers, including: ANB-NOS, BMPS, EMCS, GMBS, LC-SPDP, MBS, PDPH, SBA, SIA, Sulfo-SIA, SMPH, SPDP, Sulfo-LC-SPDP, Sulfo-MBS, Sulfo-SANPAH, and/or Sulfo-MSCC (www.proteochem.com/proteincrosslinkersheterobifunctionalcrosslinkers-c-1_7.html?gclid=CIWr7pS42tUCFUtNfgodO_sB3A).

Preferably, the cell membrane penetrating moiety comprises a cationic peptide. Cationic peptides include between 4 and 20 arginine and/or lysine residues, and preferably 9 arginine and 9 lysine residues. Other delivery methods are also contemplated such as liposomal or hydrogel formulations. In such formulations, the cell membrane penetrating moiety may be coupled to the liposome or hydrogel rather than to (or in addition to) the exogenous Tβ4, Sox2, and/or Oct 4. This discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

Coupling the exogenous Tβ4, the exogenous Sox2, and/or the exogenous Oct4 to a nuclear targeting moiety is likely to increase expression of the target genes (e.g., Oct3/4, Rex1, Nanog, Gata6, Msx2, Pax6, Hand1) responsible for reprogramming the somatic/non-embryonic cells to revert to a pluripotent states. A preferred nuclear targeting moiety is glutathione-S-transferase (e.g., UniProt database accession number P28161). In such liposomal and/or hydrogel formulations, the nuclear targeting moiety may be coupled to the liposome or hydrogel rather than to (or in addition to) the exogenous Tβ4, Sox2, and/or Oct 4.

The exogenous proteins can be produced using recombinant techniques known in the art. The inventor contemplates that the Tβ4, Sox2, and Oct4 can be translated from a single expression sequence. Alternatively, expression sequences can comprise Tβ4 and Sox2, Tβ4 and Oct4, Sox2 and Oct4, Tβ4, Sox2, Oct4 and any combination thereof. Production of the exogenous proteins may be accomplished using prokaryotic (e.g., E. coli), yeast (e.g., Saccharomyces, Pichia, Kluyveromyces, Hansenula, and Yarrowia) or eukaryotic cells (e.g., HEK293 and CHO). Insect cell and cell-free methods are also contemplated.

The inventors contemplate that induced pluripotent stem cells according to the inventive subject matter will maintain pluripotency over at least 5 passages, at least 10 passages, at least 20 passages, at least 50 passages, and preferably at least 100 passages.

The inventive subject matter also includes a composition that comprises exogenous Tβ4, exogenous Sox2, and exogenous Oct4 that can induce pluripotency in non-embryonic/somatic cells. In contemplated compositions, at least two of the exogenous Tβ4, the exogenous Sox2, and the exogenous Oct4 are coupled by a linker. In other words, a linker couples Tβ4 and Sox2, Tβ4 and Oct4, or Sox2 and Oct4. Linkers could also be used to couple Tβ4, Sox2, and Oct4.

A cell membrane penetrating moiety (e.g., a cationic peptide) and/or a nuclear targeting moiety (e.g., glutathione-S-transferase) can be coupled to the exogenous Tβ4, the exogenous Sox2, and/or the exogenous Oct4. A nuclear targeting moiety can optionally is coupled to at least one of the exogenous Tβ4; the exogenous Sox2; and the exogenous Oct4.

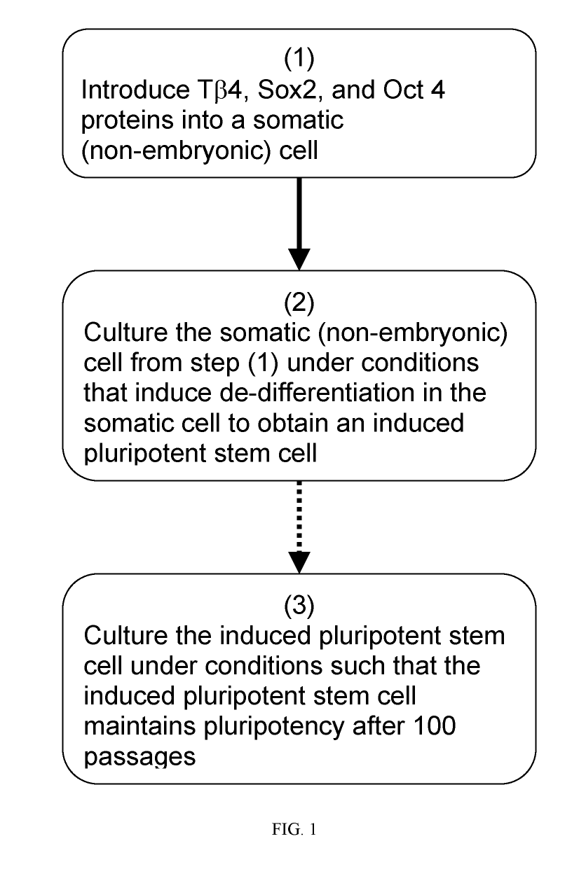

FIG. 1 shows a flow chart for a method of inducing pluripotency in a somatic (non-embryonic) cell. In step (1), Tβ4, Sox2, and Oct 4 proteins are introduced into the somatic cell. In step (2) the somatic cell from step (1) is cultured under conditions that induce de-differentiation in the somatic cell to obtain an induced pluripotent stem cell. In an optional step (3), the induced pluripotent stem cell is cultured under conditions such that the induced pluripotent stem cell maintains pluripotency after 10, 20, 50 or 100 passages.

### EXAMPLE 1

Transfect fibroblasts or adipocytes with 12 mg/ml recombinant tagged proteins every 12 hours for 28 days to reprogram cells. Culture on feeder cells with EM medium, preferably conditioned EM medium. Pick iPS colonies, expand. See e.g., Kejin Hu, All Roads Lead to Induced Pluripotent Stem Cells: The Technologies of iPSC Generation, 23(12) STEM CELLS AND DEVELOPMENT 1285-1300 (2014); Xiao-Yue Deng et al., Non-Viral Methods For Generating Integration-Free, Induced Pluripotent Stem Cells, 10 CURRENT STEM CELL RESEARCH & THERAPY 153-58 (2015).

Transformed iPSCs would be stained with alkaline phosphatase to detect expression of pluripotent stem cell markers, such as SSEA-1, SSEA-3, SSEA-4, TRA-1-60, TRA-1-81, TRA-2-49/6E, and/or Nanog.

Reprogramming can further be tested by testing for formation of embryoid bodies and or teratomas. Kazutoshi Takahashi et al., Induction of Pluripotent Stem Cells from Adult Human Fibroblasts by Defined Factors, 131 CELL 861-72 (2007). Induction of pluripotentcy can also be confirmed by generating chimeric mice. Csilla Nemes et al., Generation of Mouse Induced Pluripotent Stem Cells by Protein Transduction, 20(5) TISSUE ENGINEERING: PART C 383-392 (2014).

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. An induced pluripotent stem cell comprising:
exogenous Tβ4;
exogenous Sox2; and
exogenous Oct4.

2. The induced pluripotent stem cell of claim 1, wherein the induced pluripotent stem cell comprises a simian induced pluripotent stem cell.

3. The induced pluripotent stem cell of claim 2, wherein the induced pluripotent stem cell comprises a human induced pluripotent stem cell.

4. The induced pluripotent stem cell of claim 1, wherein the simian induced pluripotent stem cell comprises a murine induced pluripotent stem cell.

5. The induced pluripotent stem cell of claim 4, wherein the rodent induced pluripotent stem cell comprises a mouse induced pluripotent stem cell.

6. The induced pluripotent stem cell of claim 1, wherein the exogenous Tβ4 is phosphorylated.

7. The induced pluripotent stem cell of claim 1, wherein at least one of the exogenous Tβ4, the exogenous Sox2, and the exogenous Oct4 is coupled with a cell membrane penetrating moiety.

8. The induced pluripotent stem cell of claim 7, wherein the cell membrane penetrating moiety comprises a cationic peptide.

9. The induced pluripotent stem cell of claim 8, wherein the cationic peptide has a sequence consisting essentially of RRRRRRRRRKKKKKKKKK.

10. The induced pluripotent stem cell of claim 1, wherein at least one of the exogenous Tβ4, the exogenous Sox2, and the exogenous Oct4 is coupled with a nuclear targeting moiety.

11. The induced pluripotent stem cell of claim 10, wherein the nuclear targeting moiety comprises glutathione-S-transferase.

12. The induced pluripotent stem cell of claim 1, wherein the induced pluripotent stem cell maintains pluripotency over at least 100 passages.

13. A composition for inducing pluripotency in a non-embryonic cell comprising: exogenous Tβ4; exogenous Sox2; and exogenous Oct4.

14. The composition for inducing pluripotency in a non-embryonic cell of claim 13, wherein at least two of the exogenous Tβ4; the exogenous Sox2; and the exogenous Oct4 are coupled by a linker.

15. The composition for inducing pluripotency in a non-embryonic cell of claim 13, wherein a cell membrane penetrating moiety is coupled to at least one of the exogenous Tβ4; the exogenous Sox2; and the exogenous Oct4.

16. The composition for inducing pluripotency in a non-embryonic cell of claim 15, wherein the cell membrane penetrating moiety comprises a cationic peptide.

17. The composition for inducing pluripotency in a non-embryonic cell of claim 13, wherein a nuclear targeting moiety is coupled to at least one of the exogenous Tβ4; the exogenous Sox2; and the exogenous Oct4.

18. The composition for inducing pluripotency in a non-embryonic cell of claim 17, wherein the nuclear targeting moiety comprises glutathione-S-transferase.

19. A method of inducing pluripotency in a somatic cell comprising:
(1) introducing into the somatic cell Tβ4, Sox2, and Oct 4 proteins; and
(2) culturing the somatic cell from step (1) under conditions that induce de-differentiation in the somatic cell, wherein an induced pluripotent stem cell is obtained.

20. The method of claim 19, further comprising a step of culturing the induced pluripotent stem cell under conditions such that the induced pluripotent stem cell maintains pluripotency after 100 passages.
